(19) United States
(12) Patent Application Publication
(10) Pub. No.: US 2016/0252521 A1
(43) Pub. Date: Sep. 1, 2016

(54) METHOD FOR DETERMINING HIGH-MANNOSE GLYCANS

(71) Applicant: GENOVIS AB, Lund (SE)

(72) Inventors: Fredrik OLSSON, Kavlinge (SE); Stephen Georg BJOK, Helsingborg (SE); Maria NORDGREN, Hassleholm (SE); Sarah FREDRIKSSON, Dalby (SE)

(73) Assignee: Genovis AB, Lund (SE)

(21) Appl. No.: 15/030,367

(22) PCT Filed: Oct. 17, 2014
(86) PCT No.: PCT/EP2014/072364

§ 371 Date: Apr. 18, 2016

(30) Foreign Application Priority Data
Oct. 18, 2013 (GB) 1318490.8

(51) Int. Cl. G01N 33/68

(52) U.S. Cl. CPC G01N 33/6857; G01N 2333/924

(57) ABSTRACT

The present invention relates to methods for analysing molecules comprising immunoglobulin Fc regions for the presence or absence of high-mannose glycans, and to for carrying out such methods.

### FIELD OF THE INVENTION

The present invention relates to methods for analysing molecules comprising immunoglobulin Fc regions for the presence or absence of high-mannose glycans, and to kits for carrying out such methods.

### BACKGROUND OF THE INVENTION

Immunoglobulin Fc regions are integral parts of important molecules such as antibodies and Fc-fusion proteins. The characterisation of Fc regions, including structural characterisation and physiochemical analysis, is required by developers and producers of products that comprise such regions, such as antibody-based therapeutics. As well as the primary structure, it is particularly important to assess the glycan structures of an Fc region. For example, it is established that antibody molecules with Fc regions containing high-mannose glycans are cleared more rapidly in humans than molecules containing other glycan forms. High-mannose glycans are very rare on natively produced Fc regions, but as many as 20% of the IgG molecules in a batch of commercially-produced antibody can contain such structures. Accordingly this can have a significant impact on the pharmacokinetic properties of a therapeutic molecule. The high-mannose glycan content of Fc regions is therefore an important product quality attribute and there is a need for reliable techniques to assess this attribute.

### SUMMARY OF THE INVENTION

The present invention provides a method for determining the presence or absence of high-mannose glycans attached to a molecule comprising an immunoglobulin Fc region, the method comprising:

(a) contacting a sample of said molecule with an endoglycosidase polypeptide capable of cleaving high-mannose glycans; and
(b) analysing the resulting mixture for the presence or absence of high-mannose glycans and/or Fc regions containing high-mannose glycans; and optionally
(c) quantifying the proportion of molecules in the sample which contain high-mannose glycans by calculating the quantity of high-mannose glycans in the mixture relative to the total quantity of glycans in the mixture.

The invention also provides a method for determining the presence or absence of high-mannose glycans attached to a molecule comprising an immunoglobulin Fc region, the method comprising:

(a) contacting a first portion of a sample of said molecule with an endoglycosidase polypeptide capable of cleaving high-mannose glycans;
(b) analysing the resulting mixture for the presence or absence of high-mannose glycans and/or Fc regions containing high-mannose glycans;
(c) contacting a second portion of said sample with an endoglycosidase polypeptide not capable of cleaving high-mannose glycans;
(d) analysing the resulting mixture for the presence or absence of high-mannose glycans and/or Fc regions containing containing high-mannose glycans; and
(e) comparing the results of the analysis in (b) to the results of the analysis in (d) to thereby determine the presence or absence of molecules in the sample which contain high-mannose glycans.

The invention also provides a kit comprising a polypeptide comprising or consisting of the sequence of SEQ ID NO: 1, or a variant thereof, and optionally (a) means for detecting high-mannose glycans and/or high-mannose containing IgG molecules; and/or (b) instructions to detect high-mannose glycans and/or high-mannose containing IgG molecules. The kit may also comprise a polypeptide comprising or consisting of the sequence of SEQ ID NO: 2, or a variant thereof.

### BRIEF DESCRIPTION OF THE FIGURES

FIG. 1 shows results from SDS-PAGE following treatment of the human monoclonal antibodies Cetuximab (A), Adalimumab (B), Panitumumab (C) and Denosumab (D) with either EndoS and IdeS, EndoS49 and IdeS, or IdeS alone. Untreated controls are also shown.

FIG. 2 shows the UHPLC chromatograms for Cetuximab (A), Adalimumab (B), Panitumumab (C) and Denosumab (D) either untreated, or after treatment with either EndoS or EndoS49. All mAbs were digested with IdeS for enhanced resolution of Fc fragments before running the UHPLC.

FIG. 3 shows the results of MALDI-TOF analysis of the glycans released following treatment of each of Cetuximab, Adalimumab, Panitumumab and Denosumab with either EndoS or EndoS49.

FIG. 4 summarises the results of the analysis of Cetuximab, Adalimumab, Panitumumab and Denosumab.

FIG. 5 shows typical glycan structures found attached to the Fc region of IgG.

FIG. 6 shows high-mannose glycan structures which may be found attached to Fc regions.

### BRIEF DESCRIPTION OF THE SEQUENCES

SEQ ID NO:1 is the amino acid sequence of the EndoS49 polypeptide isolated from S. pyogenes M49 serotype NZ131. Sequence also available under Genbank Accession no. ACI61688.1.

SEQ ID NO:2 the amino acid sequence of the EndoS polypeptide isolated from S. pyogenes AP1.

SEQ ID NO:3 is the amino acid sequence of the IdeS polypeptide isolated from S. pyogenes AP1.

SEQ ID NO:4 is the amino acid sequence of EndoS isolated from S. pyogenes AP1, including a putative signal sequence.

SEQ ID NO:5 is the amino acid sequence of IdeS isolated from S. pyogenes AP1, including a putative signal sequence.

### DETAILED DESCRIPTION OF THE INVENTION

It is to be understood that different applications of the disclosed methods and products may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. Thus, for example, reference to “a molecule” includes two or more such molecules, and the like. The terms protein and polypeptide are used interchangeably herein. The terms antibody and immunoglobulin are used interchangeably herein. All publications, patents and patent applications cited herein, whether supra or infra, are hereby incorporated by reference in their entirety.

The present invention relates to methods for analysing molecules comprising immunoglobulin Fc regions for the presence or absence of high-mannose glycans. Immunoglobulin Fc regions are glycoproteins, which means that they have glycans chemically linked to some of their amino acid residues. As described herein, any glycan referred to as being “on” or “attached to” an Fc region, or to an antibody molecule, means that said glycan is linked to an amino acid of said region or molecule. Similarly, if an Fc region or antibody molecule is referred to as “containing” a glycan, this also means that said glycan is linked to an amino acid of said region molecule. The linkage of carbohydrates to proteins occurs through O-linkage or N-linkage. O-linked carbohydrates attach to the oxygen atom of the side chain of serine or threonine. N-linkage is more common and requires the attachment of a sugar to the amide nitrogen atom on the side chain of asparagine. Antibodies do not generally contain O-linked glycans, with the exception of some IgA1 and IgD molecules. Accordingly, unless otherwise specified, glycans referred to herein are N-linked.

The Fc region of an IgG molecule has a single conserved N-linked glycosylation site at Asn-297 of the γ-chain (Kabat numbering). This means that there are up to two glycans per whole IgG molecule, which are highly heterogeneous and are selected from over 30 different types, giving rise to over 400 different glyco forms. Other immunoglobulin isotypes may be even more heterogenous in their glycosylation patterns. For example human IgE has seven N-linked glycans attached to the heavy ε-chain at different sites, several of which are located in the Fc-region; Asn-265 in the Cε2 domain, and Asn-371 and Asn-394 in the Cε3 domain. In addition, IgE from non-myeloma can have a further glycan at Asn-383 in the Cε3 domain. Based on sequence alignment, Asn-265 in the Cε2 domain of IgE corresponds to Asn-297 of IgG. In fact, sequence alignment between IgG, IgD and IgE shows that the Asn-297 region on IgG is completely conserved in all three immunoglobulin isotypes, and may have a conserved role in folding, post-translational modification and function.

The particular glycan attached to a given glycosylation site on an Fc region may vary depending on how the molecule comprising the Fc region is produced, for example depending on the cell line used, or on other production conditions. Therefore, in any given batch of a molecule comprising an Fc region, such as a batch of monoclonal antibody, there may be large numbers of different glycan structures present, and furthermore this can vary from batch to batch. The methods described herein are particularly suited to the characterization of molecules comprising Fc regions for the purposes of quality control, for example to compare the proportion of high-mannose glycans present in different batches of the same molecule. This may be particularly relevant to the regulatory assessment of therapeutic or diagnostic antibodies or therapeutic or diagnostic Fc-fusion proteins.

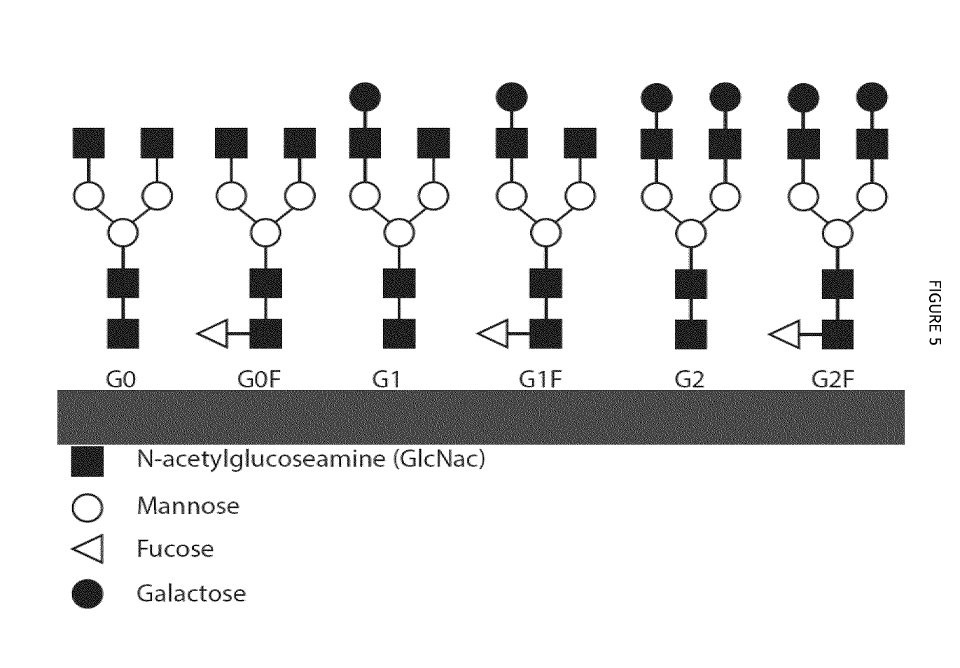

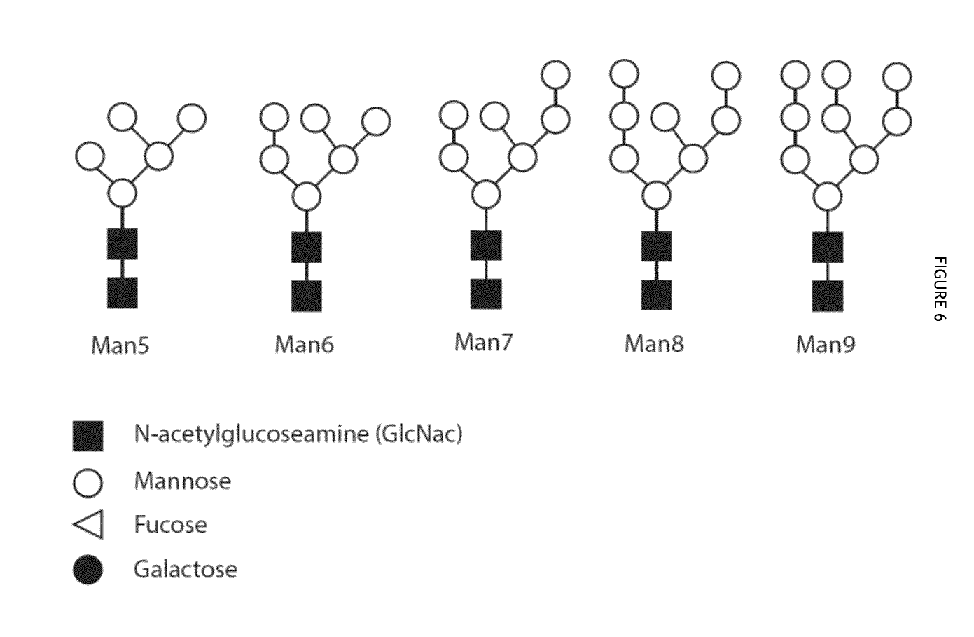

The present invention relates to various methods for analysing molecules comprising immunoglobulin Fc regions for high-mannose glycan structures. These structures are very rare on the Fc regions of immunoglobulin molecules produced in normal animals, but as many as 20% of the IgG molecules in a batch of commercially-produced antibody can contain such structures. Typical glycan structures found attached to the Fc region of IgG are shown in FIG. 5. High-mannose glycan structures are shown in FIG. 6, and are typically referred to as Man5, Man6, Man7 and Man8.

Any suitable sample containing molecules comprising immunoglobulin Fc regions may be assessed by the methods described herein. The sample is typically a fluid. For example, the sample may be a blood, serum or saliva sample. Alternatively the sample may be taken from a batch of synthetically produced molecules. The sample may be formulated for administration to a patient with a pharmaceutical carrier or diluent.

The Fc region may be from any species, for example, human, monkey, rabbit, sheep or mouse, but preferably human. The Fc region may be of any isotype selected from IgG, IgE, IgA, IgD or IgM, preferably IgG. The Fc region may be of mouse subclass IgG2a or IgG3. Preferably, the Fc region may be of human subclass IgG1, IgG2, IgG3 or IgG4.

The molecule comprising an immunoglobulin Fc region may be a whole antibody or may be a fragment thereof comprising the Fc region. Such an antibody may be monoclonal or polyclonal. The antibody may be human, humanised or chimeric. Examples of suitable antibodies include denosumab, adalimumab, panitumumab, cetuximab, trastazumab and bevacizumab. The antibody may be bispecific, and/or may comprise another chemical moiety conjugated to the Fc region. For example, the antibody may be an antibody-drug conjugate. An antibody-drug conjugate may comprise antibody conjugated to a therapeutic agent which is a cytotoxin. Suitable toxins include avristatin, calicheamicins, CC-1065, doxorubicin, maytonsinoid, methotrexate and vinca alkaloids.

Alternatively, the molecule comprising an immunoglobulin Fc region may be an Fc-fusion protein, that is an immunoglobulin Fc region covalently linked to another polypeptide. Examples of suitable Fc-fusions include Etanercept.

Disclosed herein is a method for determining the presence or absence of high-mannose glycans attached to a molecule comprising an immunoglobulin Fc region, the method comprising:

(a) contacting a sample of said molecule with an endoglycosidase polypeptide capable of cleaving high-mannose glycans; and
(b) analysing the resulting mixture for the presence or absence of high-mannose glycans and/or Fc regions containing high-mannose glycans; and optionally
(c) quantifying the proportion of molecules in the sample which contain high-mannose glycans by calculating the quantity of high-mannose glycans in the mixture relative to the total quantity of glycans in the mixture.

The quantification in step (c) will typically comprise measuring the quantity of detectable, free high-mannose glycans following step (a), also measuring the quantity of other detectable, free glycan forms following step (a) and comparing the respective quantities. The proportion of molecules which originally contained high-mannose glycans can thereby be determined.

Optionally, prior to step (a) the sample may be contacted with an endoglycosidase polypeptide not capable of cleaving high-mannose glycans, optionally wherein molecules comprising Fc regions are isolated from the resulting mixture and used in step (a). This step may be particularly helpful if high-mannose glycans detected following step (a) are to be isolated and further characterised, because other glycan types will largely have been removed. Such isolation and characterisation is also encompassed by the methods described herein.

Where the molecule to be analysed comprises an immunoglobulin Fc region of isotype IgG, for example if the molecule is an IgG antibody, the resulting mixture from (a) may optionally be contacted with an IgG cysteine protease prior to the analysis in step (b). The IgG cysteine protease will typically cleave the Fc region in the hinge, resulting in a separate Fc fragment. This fragment may be isolated from the resulting mixture and then analysed in step (b). Isolation of the Fc fragment may facilitate the subsequent analysis.

In any method described herein, above or below, by “analysing the resulting mixture for the presence or absence of high-mannose glycans and/or Fc regions containing high-mannose glycans” it is meant that any suitable method may be applied for the detection of the indicated entity. Detection may typically be based on molecular weight. Suitable methods for detecting glycans in solution may include HPAE (high performance anion-exchange), (U)HPLC ((ultra)high performance liquid chromatorgraphy) and mass spectrometry. Mass spectrometry, (U)HPLC or SDS-PAGE may be used to detect molecules comprising Fc regions containing high-mannose glycans. Detection of a glycan (attached to another molecule or free) may be enhanced by labelling with any suitable label. For example, a fluorophore such as 2-AB (2-aminobenzamide) may be used. The glycans may be purified to remove protein, peptides, salts, detergents, and any additional contaminating substances prior to labelling.

Regardless of the specific detection method used, high mannose-glycans and molecules comprising Fc regions containing high-mannose glycans may also be identified by comparing the results of contacting a portion of a sample with an endoglycosidase polypeptide which is capable of cleaving high-mannose glycans with the results of contacting a portion of the same sample with an endoglycosidase polypeptide which is not capable of cleaving high-mannose glycans, for example as set out in the following.

Also disclosed herein is a method for determining the presence or absence of high-mannose glycans attached to a molecule comprising an immunoglobulin Fc region, the method comprising:

(a) contacting a first portion of a sample of said molecule with an endoglycosidase polypeptide capable of cleaving high-mannose glycans;
(b) analysing the resulting mixture for the presence or absence of high-mannose glycans and/or Fc regions containing high-mannose glycans;
(c) contacting a second portion of said sample with an endoglycosidase polypeptide not capable of cleaving high-mannose glycans;
(d) analysing the resulting mixture for the presence or absence of high-mannose glycans and/or Fc regions containing containing high-mannose glycans; and
(e) comparing the results of the analysis in (b) to the results of the analysis in (d) to thereby determine the presence or absence of molecules in the sample which contain high-mannose glycans.

Where the molecule comprises an immunoglobulin Fc region of isotype IgG, for example if the molecule is an IgG antibody, the resulting mixture from (a) may optionally be contacted with an IgG cysteine protease prior to the analysis in step (b), and/or the resulting mixture from (c) may be contacted with an IgG cysteine protease prior to the analysis in step (d). The IgG cysteine protease will typically cleave the Fc region in the hinge, resulting in a separate Fc fragment. This fragment may be isolated from the resulting mixture and then analysed in step (b) or (d), respectively. Isolation of said Fc fragment may facilitate the subsequent analysis.

Determining the presence or absence of molecules in the sample which contain high-mannose glycans in step (e) may be achieved by any suitable method. For example, free high-mannose glycans will be present in the mixture resulting from step (a), but absent in the mixture resulting from step (c). Thus, the presence of high-mannose glycans can be determined by detecting an entity (peak, band etc) in the mixture resulting from step (a) which is absent in the mixture resulting from step (c). Conversely, Fc regions containing high-mannose glycans will be absent from the mixture resulting from step (a), but present in the mixture resulting from step (c). Thus, the presence of high-mannose glycans can also be determined by detecting an entity (peak, band etc) in the mixture resulting from step (c) which is absent in the mixture resulting from step (a). Step (e) may optionally further comprises quantifying the proportion of molecules in the sample which contain high-mannose glycans, for example by calculating the proportion of detected high-mannose glycans relative to the total of all detected glycans in the mixture resulting from step (a), or by calculating the proportion of Fc regions detected containing high-mannose glycans relative to the total of all detected Fc regions in the mixture resulting from step (c).

In other words, the presence or absence of an entity may be determined by subtracting the results of treatment with an endoglycosidase polypeptide capable of cleaving high-mannose glycans from the results of treatment with an endoglycosidase polypeptide not capable of cleaving high-mannose glycans (or vice versa). The proportion of that entity present in the original sample may be achieved by quantifying whatever remains following the subtraction, as a proportion of the whole. For example, if a portion of a sample of IgG is treated with an endoglycosidase polypeptide capable of cleaving high-mannose glycans and another portion of the same sample is treated with an endoglycosidase polypeptide not capable of cleaving high-mannose glycans, and the resulting mixtures from both treatments are analysed by, for example HPLC or mass spectrometry, a peak corresponding to high-mannose containing IgG can be identified by virtue of its absence in the analysis of the former portion by comparison to its presence in the analysis of the latter portion. Said peak may then be quantified relative to other peaks in the analysis of the latter portion to determine the proportion of high-mannose containing IgG in the original sample. The same reasoning could be applied to the presence or absence of particular bands in an SDS-PAGE analysis.

As will be appreciated, the methods of the invention may therefore be used to establish a characteristic glycan profile for the molecules comprising an Fc region in any given sample. This profile will typically include information regarding the proportion of molecules containing high-mannose glycans. The profile may include, for example, the identification of particular bands in SDS-PAGE analysis, or peaks in mass spectrometry or HPLC analysis of a sample, as corresponding to molecules containing particular glycan structures, particularly high-mannose glycans. Such a profile may be used to characterize a particular batch from a process for the commercial production of said molecule. Once such a profile is established, it may be used in subsequent methods for the purposes of comparison. The profile may be used in place of any of the steps of the methods described herein. For example, a method may comprise only steps contacting a sample with only a polypeptide not capable of cleaving high-mannose glycans, analyzing the resulting mixture, and comparing the results of said analysis to a glycan profile previously established by a method of the invention, to thereby determine the presence or absence of high-mannose containing IgG in the sample. Effectively such a method comprises only steps (c) and (d) above, with step (e) comprising a comparison with the established profile rather than with the results of steps (a) and (b).

In any method described herein, the step of contacting a sample with an endoglycosidase polypeptide is performed under any conditions that permit the cleavage of glycan structures on an Fc region by the specific endoglycosidase polypeptide that is used. Similarly, where an IgG cysteine protease is used, the relevant step may be performed under any conditions that permit the cleavage of target molecules by the protease. Suitable conditions are described in the Examples. Typically, any standard buffer is used at a pH of 6.5 to 8.0. Standard buffers include phosphate buffer saline (PBS), tris, ammonium bicarbonate, MES, HEPEs and sodium acetate. Typically, a sample is incubated with a polypeptide for at least 20 minutes, at least 30 minutes, at least 40 minutes, at least 50 minutes, preferably at least 60 minutes. Incubation preferably takes place at room temperature, more preferably at approximately 20° C., 25° C., 30° C., 35° C., 40° C. or 45° C., and most preferably at approximately 37° C. The endoglycosidase polypeptides or cysteine proteases may be immobilised on solid supports for use in the methods described herein. Suitable solid supports include agarose beads, silica beads, poly-styrene, divinyl benzene or combinations thereof. Commercially available examples are Sepharose (GE heathcare, POROS (Life Technlogies), or similar resins, provided a protein can be conjugated to the surface.

The methods disclosed herein utilise endoglycosidase polypeptides which hydrolyse N-linked glycan structures attached to immunoglobulin Fc regions. In other words, said polypeptides cleave said glycan structures from the molecule to which they are attached. For example, the endoglycosidate polypeptides typically hydrolyse N-linked glycan structures attached to Asn-297 of the γ-chain of an Fc region of isotype IgG (Kabat numbering). The endoglycoside polypeptides used herein may be endoglycosidase polypeptides which are capable of cleaving high-mannose glycans attached to an immunoglobulin Fc region, or may be endoglycosidase polypeptides which are not capable of cleaving high-mannose glycans attached to an immunoglobulin Fc region, as specified below.

Endoglycosidase from serotype M49 Streptococcus pyogenes, referred to herein as EndoS49 or EndoS2, was isolated from strain NZ131, a nephritogenic and highly transformable strain of serotype M49. NZ131 strain is a clinical isolate from a case of acute post-streptococcal glomerulonephritis in New Zealand. The inventors have determined that EndoS49 is capable of cleaving high-mannose glycans attached to the Fc region of IgG, as well as other typical glycan structures found on IgG. The amino acid sequence of EndoS49 is shown in SEQ ID NO: 1. An endoglycosidase capable of cleaving high-mannose glycans may comprise or consist of the sequence shown in SEQ ID NO: 1, or a variant thereof. A variant of SEQ ID NO: 1 is a polypeptide that has an amino acid sequence which varies from that of SEQ ID NO: 1 and which retains its functional characteristics. Specifically it has endoglycosidase activity, including the ability to cleave high-mannose glycans. The endoglycosidase activity of a variant, and its ability to cleave high-mannose glycans can be assayed using any method known in the art.

For example, a test polypeptide may be incubated with a sample of IgG molecules at a suitable temperature, such as 37° C. The starting materials and reaction products may then be analysed by SDS-PAGE to determine whether all or only some glycans on the IgG have been hydrolysed. If all glycans have been hydrolysed a single fraction of ˜150 kDa will be visible, corresponding to deglycosylated IgG. If only some glycans are hydrolysed, there will be two different fractions of ˜150 kDa, a heavier fraction which corresponds to glycosylated IgG and a lighter fraction which corresponds to deglycosylated IgG. The presence of a glycosylated IgG fraction in this context may indicate that high-mannose glycans have not been cleaved. Alternatively, following incubation of a test polypeptide with IgG the resulting mixture may be directly assessed, for example using mass spectrometry, to determine whether the test polypeptide has hydrolysed high-mannose glycans, which can be distinguished from other glycan structures present in the resulting mixture by molecular weight. Suitable methods are also described in the Examples.

Endoglycosidase from S. pyogenes API, referred to herein as EndoS, was isolated from S. pyogenes API. The amino acid sequence of EndoS is provided as SEQ ID NO: 2. The inventors have also determined that EndoS is not capable of cleaving high-mannose glycans attached to IgG, but does cleave other typical glycan structures found on IgG. The amino acid sequence of EndoS is shown as SEQ ID NO: 2. Also shown as SEQ ID NO: 4 is the sequence of EndoS including a putative signal sequence. An endoglycosidase that is not capable of cleaving high-mannose glycans may comprise or consist of the sequence shown in SEQ ID NO: 2 or 4, or a variant thereof. A variant of SEQ ID NO: 2, is a polypeptide that has an amino acid sequence which varies from that of SEQ ID NO: 2 and which retains its functional characteristics. Specifically it has endoglycosidase activity, but lacks the ability to cleave high-mannose glycans. The same considerations apply to a variant of SEQ ID NO: 4. The endoglycosidase activity of a variant, and its ability to cleave high-mannose glycans can be assayed using any method known in the art, for example as described above.

At a protein level, EndoS is 108 kDa compared to the 90 kDa of EndoS49. EndoS49 has less than 40% identity to EndoS. Both polypeptides have a family 18 glycoside hydrolase catalytic domain. In EndoS49, this active site corresponds to residues 179 to 186 of SEQ ID NO: 1. In EndoS, the active site corresponds to residues 191 to 199 of SEQ ID NO: 2. In both proteins, these residues include the motif D**D*D*E. The glutamic acid at position 186 of SEQ ID NO: 1 is essential for enzymatic activity in EndoS49. The glutamic acid at position 199 of SEQ ID NO: 2 is essential for enzymatic activity in EndoS.

The methods disclosed herein may optionally also utilize IgG cysteine protease polypeptides. A typical IgG cysteine protease polypeptide is the Immunoglobulin degrading enzyme from S. pyogenes, referred to herein as IdeS. IdeS was isolated from S. pyogenes API. The sequence of IdeS is shown as SEQ ID NO: 3. Also shown as SEQ ID NO: 5 is the sequence of IdeS including a putative signal sequence. An IgG cysteine protease polypeptide may comprise or consist of the sequence shown in SEQ ID NO: 3 or 5, or a variant thereof. A variant of SEQ ID NO: 3 is a polypeptide that has an amino acid sequence which varies from that of SEQ ID NO: 3 and which retains its functional characteristics. Specifically it has IgG cysteine protease activity cleaving IgG at the hinge region, typically between positions 249 and 250 according to the Kabat numbering system. The same considerations apply to a variant of SEQ ID NO: 5. The IgG cysteine protease activity of a variant can be assayed using any method known in the art.

For example, a test polypeptide may be incubated with a sample of IgG at a suitable temperature, such as 37° C. The starting materials and reaction products may then be analysed by SDS-PAGE to determine whether the desired IgG cleavage product is present. The cleavage product may be subjected to N-terminal sequencing to verify that cleavage has occurred in the hinge region of IgG. The cysteine protease activity of the polypeptide can be further characterised by inhibition studies. Preferably, the activity is inhibited by the peptide derivative Z-LVG-CHN₂ and/or by iodoacetic acid both of which are protease inhibitors. Preferably the activity is generally not inhibited by E64. Suitable methods are described in the Examples.

Over the entire length of the amino acid sequence of SEQ ID NO: 1 a variant of SEQ ID NO: 1 will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 1 over the entire sequence. There may be at least 80%, for example at least 85%, 90% or 95%, amino acid identity over a stretch of 100 or more, for example 125, 150, 200, 250, 300, 400, 500, 600, 700 or 800 or more, contiguous amino acids (“hard homology”). The same considerations in respect of % identity also apply to variants of SEQ ID NOs: 2, 3, 4 or 5.

Standard methods in the art may be used to determine homology. For example the UWGCG Package provides the BESTFIT program which can be used to calculate homology, for example used on its default settings (Devereux et at (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et at (1990) J Mol Biol 215:403-10. Software for performing BLAST analyses is publicly available through the National Center for Biotechnology Information (http://www.ncbi.nlm.nih.gov/).

A variant of SEQ ID NO: 1 may include the substitution, deletion or insertion of single amino acids or groups of amino acids relative to the sequence of SEQ ID NO: 1. For example up to 1, 2, 3, 4, 5, 10, 20 or 30 substitutions, deletions or insertions. Substitutions are preferably conservative substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A below. Where amino acids have similar polarity, this can also be determined by reference to the hydropathy scale for amino acid side chains in Table B below. The same considerations in respect of substitutions, deletions or insertions also apply to variants of SEQ ID NOs: 2, 3, 4 or 5.

TABLE A
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, neutral |
| Asp | polar, hydrophilic, charged | Pro | hydrophobic, neutral |
| | (−) | | |
| Glu | polar, hydrophilic, charged | Gln | polar, hydrophilic, neutral |
| | (−) | | |
| Phe | aromatic, hydrophobic, | Arg | polar, hydrophilic, |
| | neutral | | charged (+) |
| Gly | aliphatic, neutral | Ser | polar, hydrophilic, neutral |
| His | aromatic, polar, hydro- | Thr | polar, hydrophilic, neutral |
| | philic, charged (+) | | |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, hydro- |
| | neutral | | phobic |

TABLE B
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Variants of SEQ ID NO: 1 may include fragments of SEQ ID NO: 1. Such fragments retain the functional characteristics of the polypeptide of SEQ ID NO: 1. A fragment may be at least 50, 100, 150, 200, 250, 300, 400 or 500 amino acids in length. A fragment may be up to 100, 200, 250, 300, 500, 750, 800 or 840 amino acids in length. Any of the above lower limits may be combined with any of the above upper limits to provide a range for the size of a fragment. For example the fragment may be between 150 and 800 amino acids in length. Preferably, the fragment encompasses residues 179 to 186 of SEQ ID NO: 1, i.e. the active site of EndoS49.

Variants of SEQ ID NO: 2 or 4 may include fragments of each respective sequence. Such fragments retain the functional characteristics of the polypeptide of SEQ ID NO: 2. A fragment may be at least 50, 100, 150, 200, 250, 300, 400 or 500 amino acids in length. A fragment may be up to 100, 200, 250, 300, 500, 750, 800, 850, 900, 950 or 955 amino acids in length, Any of the above lower limits may be combined with any of the above upper limits to provide a range for the size of a fragment. For example the fragment may be between 150 and 800 amino acids in length. Preferably, the fragment encompasses residues 191 to 199 of SEQ ID NO: 2, i.e. the active site of EndoS. A preferred fragment consists of amino acids 1 to 409 of SEQ ID NO: 2, which corresponds to the enzymatically active α-domain of EndoS generated by cleavage by the streptococcal cysteine proteinase SpeB.

Variants of SEQ ID NO: 3 or 5 may include fragments of each respective sequence. Such fragments retain the functional characteristics of the polypeptide of SEQ ID NO: 3. A fragment may be at least 50, 100, 150 or 200, 250 or 300 amino acids in length. A fragment may be up to 100, 200, 250 or 300 amino acids in length, Any of the above lower limits may be combined with any of the above upper limits to provide a range for the size of a fragment. For example the fragment may be between 200 and 300 amino acids in length.

Variants of SEQ ID NOs: 1, 2, 3, 4 or 5 may include homologous polypeptides from another organism, such as another Streptococcus bacterium, for example Streptococcus equi, Streptococcus zooepidemicus or, preferably, another Streptococcus pyogenes strain, provided that said homologue retains the functional characteristics of the respective original polypeptide. For example, a variant of SEQ ID NO: 2 may be from Corynebacterium pseudotuberculosis, for example the CP40 protein; from Enterococcus faecalis, for example the EndoE protein; or from Elizabethkingia meningoseptica (formerly Flavobacterium meningosepticum), for example the EndoF₂ protein.

The amino acid sequence of any polypeptide or variant as described herein may be modified to include non-naturally occurring amino acids and/or to increase the stability of the compound. When the polypeptides are produced by synthetic means, such amino acids may be introduced during production. The polypeptides may also be modified following either synthetic or recombinant production. The polypeptides, variants or fragments described herein may be produced using D-amino acids. In such cases the amino acids will be linked in reverse sequence in the C to N orientation. This is conventional in the art for producing such polypeptides. A number of side chain modifications are known in the art and may be made to the side chains of the polypeptides, variants or fragments, subject to their retaining any further required activity or characteristic as may be specified herein. It will also be understood that the polypeptides or variants may be chemically modified, e.g. post-translationally modified. For example, they may be glycosylated, phosphorylated or comprise modified amino acid residues.

Also described herein are kits, which may typically be used for carrying out the methods of the invention. A kit may comprise an endoglycosidase polypeptide capable of cleaving high-mannose glycans, and optionally (a) means for detecting high-mannose glycans and/or high-mannose containing Fc regions; and/or (b) instructions to detect high-mannose glycans and/or high-mannose containing Fc regions. Alternatively a kit may comprise an endoglycosidase polypeptide capable of cleaving high-mannose glycans and an endoglycosidase polypeptide not capable of cleaving high-mannose glycans, and optionally (a) means for detecting high-mannose glycans and/or high-mannose containing Fc regions; and/or (b) instructions to detect high-mannose glycans and/or high-mannose containing Fc regions. Means for detecting high-mannose glycans include labels, particularly fluorophores, which bind to glycans. An example of a suitable fluorophore is 2-aminobenzamide (2-AB).

The following Example illustrates the invention:

### Example

### Materials and Methods

### Antibodies and Enzymes

Cetuximab, Adalimumab, Panitumumab and Denosumab were obtained from the Swedish pharmacy (Apoteket AB) and used after removal of preservatives by desalting. EndoS is commercially available as IgGZERO™ and a 5000 U vial was resolved in 50 μL purified water (MilliQ™-purification). EndoS2 (i.e. EndoS49) was prepared similarly. IdeS is commercially available as FabRICATOR™ and 2000 U were dissolved in 50 μL purified water (MilliQ™-purification).

### SDS-PAGE

A sample of each mAb was incubated separately with either EndoS or EndoS2 for 30 min at 37° C. in 10 mM PBS, 150 mM NaCl, pH 7.4. IdeS was then added and co-incubated for an additional 10 min. The sample was mixed with LDS buffer and heated to 70° C. for 10 minutes and then loaded on a SDS-PAGE 4-12% Bis-Tris gel and run at 180V for 40 min using MES buffer. Ratio mAb:endoglycosidase:IdeS, 50:1:2.

### MALDI-TOF

MALDI-TOF was performed by Panatec GmbH (Heilbronn, Germany). In brief, N-glycans were released from the four mAbs by treatment with EndoS or EndoS2 for 30 min at 37° C. in 50 mM ammonium bicarbonate (NH4HCO3) pH 7.4. Ratio mAb:endoglycosidase, 10:1. The released glycan fraction was then subjected to Ultra-filtration (NanoSep 10 k Omega), before concentration of the permeate performed by Speed-Vac. MALDI-TOF analysis (positive reflector mode, DHB matrix) using a Bruker UltrafleXtreme (Bremen, Germany).

### UHPLC

Reversed phase chromatography was performed on an Agilent™ 1290 UHPLC system using an ACQUITY™ BEH 300 C4 column (1.7 um, 2.1×100 mm) from Waters. The column was conditioned in 0.1% TFA in MQ water at 65 C, 0.4 ml/min, and the antibody fragments were eluted in a slow gradient of 0.1% TFA in 60% acetonitrile/40% isopropanol. Detection was at 280 nm.

### Results

### SDS-PAGE

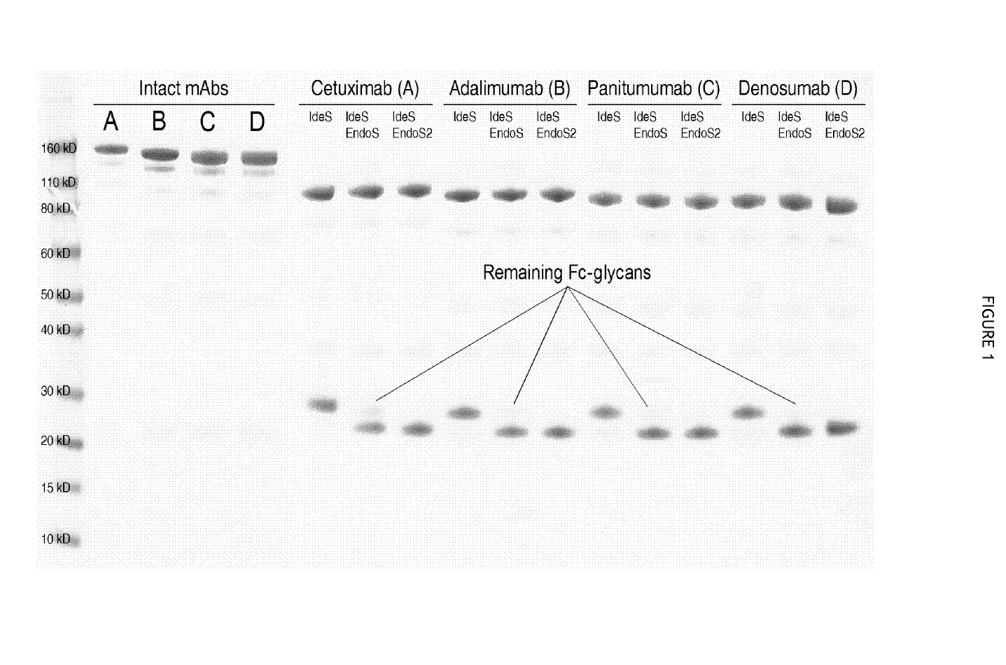

Cetuximab (A), Adalimumab (B), Panitumumab (C) and Denosumab (D) were deglycosylated using either EndoS or EndoS2 after which IdeS enzyme was added for digestion to generate scFc and F(ab′)2. The samples were analysed by SDS-PAGE and the results are shown in FIG. 1. For all four antibodies a weak band was visible at about 30 kDa when treated with EndoS but not when treated with EndoS2, indicating that EndoS2 treatment results in more complete deglycosylation.

### UHPLC

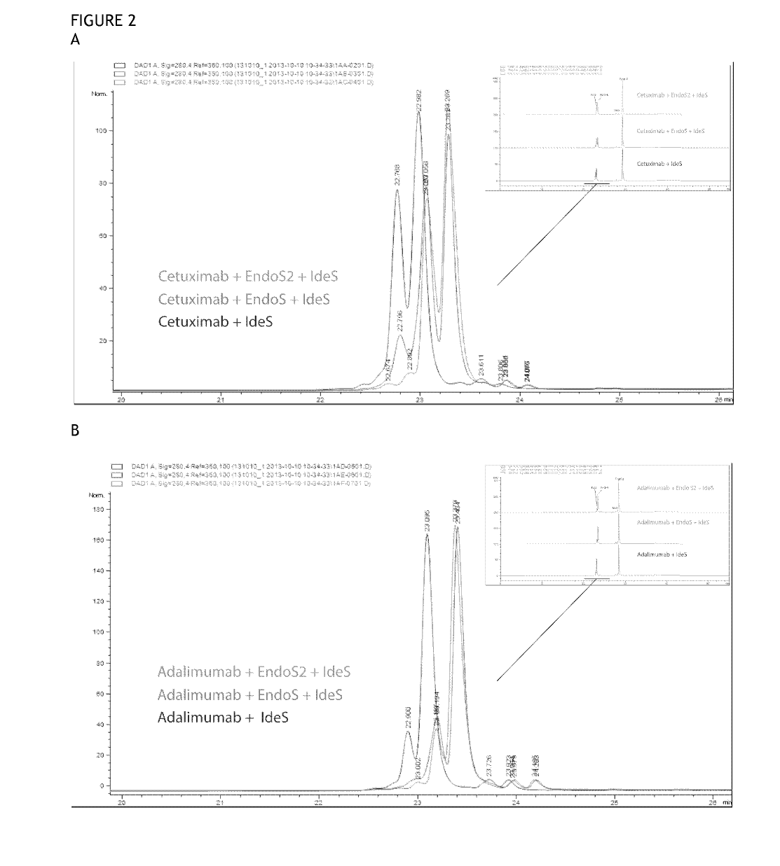

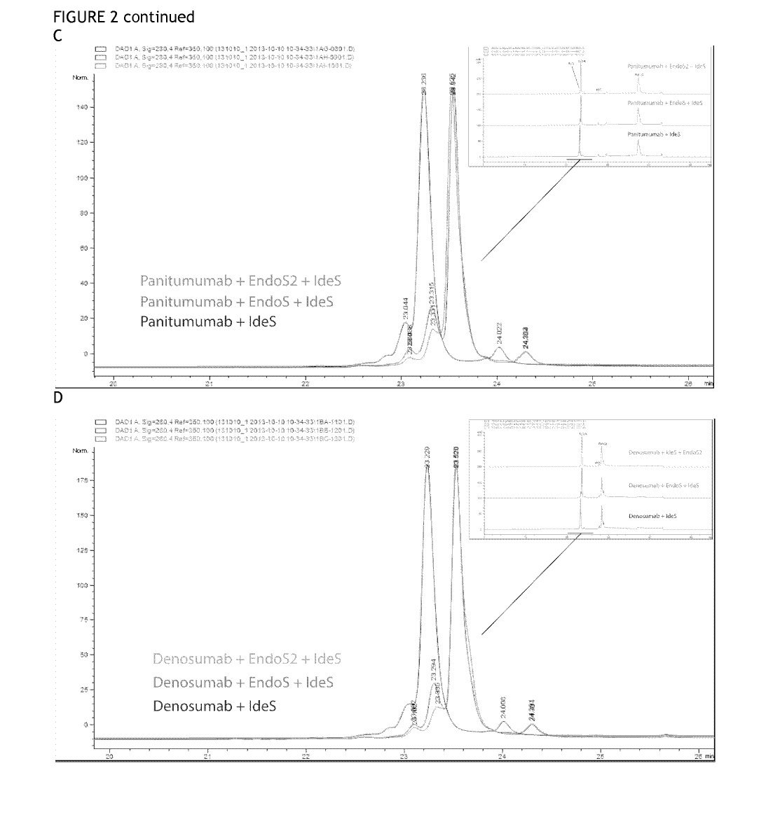

Cetuximab (A), Adalimumab (B), Panitumumab (C) and Denosumab (D) were deglycosylated using either EndoS or EndoS2, or left untreated, after which IdeS enzyme was added for digestion to generate scFc and F(ab′)2. The samples were analysed by RP-UHPLC and the results are shown in FIG. 2. Fragments were well resolved (FIG. 2 inserts) and the scFC peak was studied in more detail. A clear shift in retention time was observed when Fc-glycans were removed using EndoS and EndosS2 compared to non-deglycosaled mAbs. The chromatographic profile were highly similar when comparing EndosS2 treated mAbs with glycosylated (non-treated) Fc apart from a shift in retention time. However, the EndoS chromatogram reveals differences in number of peaks and the shape of individual peaks, indicating that EndoS and EndoS2 have differing deglycosylation effects.

### MALDI-TOF

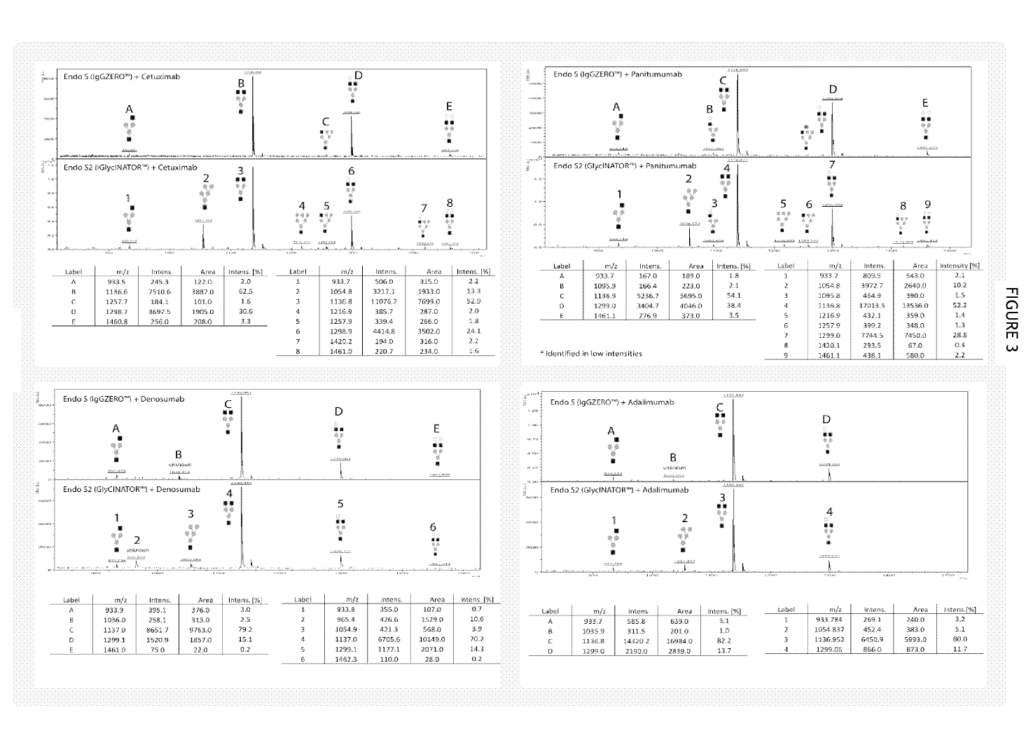

The glycans released following treatment of Cetuximab (A), Adalimumab (B), Panitumumab (C) and Denosumab with either EndoS or EndoS2 were further analyzed by MALDI-TOF. Results are shown in FIG. 3. The released glycans from treatment with EndoS2 revealed the release of high-mannose structures that were not detected when the monoclonal antibodies were treated with EndoS. Additionally, the data suggest that EndoS have lower activity on hybrid glycan structures compared to EndoS2 (Low levels of hybrid glycans were detected). By comparing the the results achieved with each enzyme, it was possible to identify the peaks corresponding to high-mannose structures, which could then be quantified.

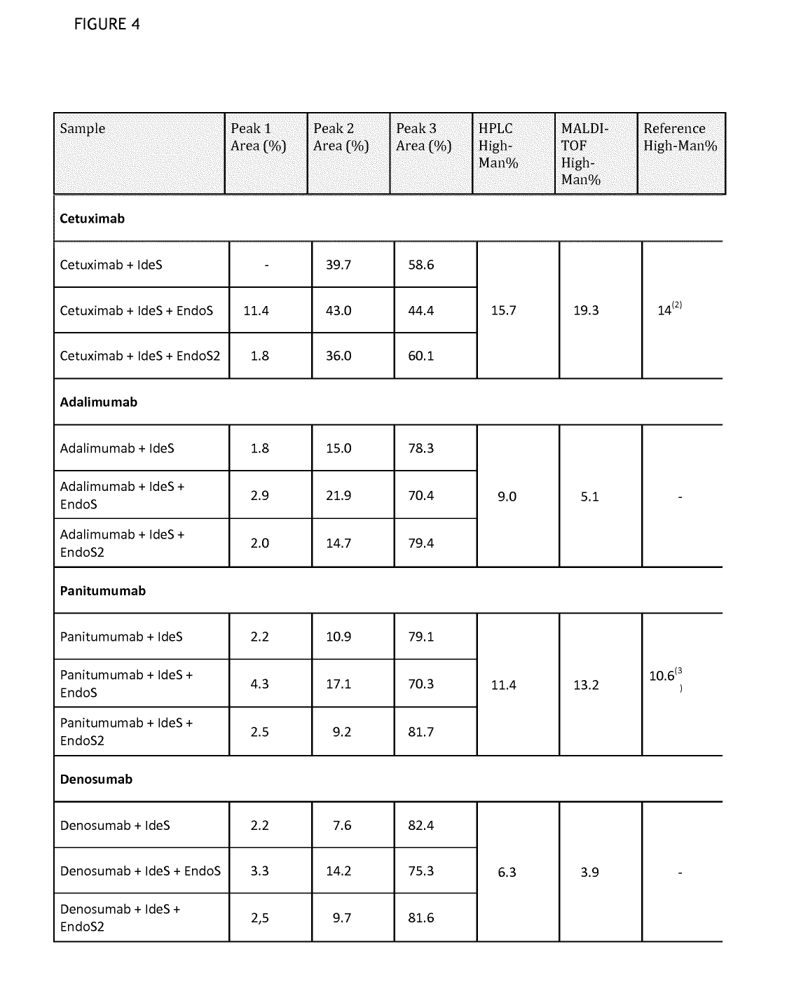

The results are summarized in FIG. 4, which indicates in particular the proportion of high-mannose glycans as a total of the detected glycan population for each antibody. Reference levels of high-mannose glycans are indicated where these were available.

### CONCLUSION

High-mannose glycans were detectable and quantifiable in samples of all four antibodies tested using the methods described herein.

1. A method for determining the presence or absence of high-mannose glycans attached to a molecule comprising an immunoglobulin Fc region, the method comprising:
(a) contacting a sample of said molecule with an endoglycosidase polypeptide capable of cleaving high-mannose glycans to form a mixture; and
(b) analysing the resulting mixture for the presence or absence of high-mannose glycans and/or Fc regions containing high-mannose glycans; and optionally
(c) quantifying a proportion of molecules in the sample which contain high-mannose glycans by calculating a quantity of high-mannose glycans in the mixture relative to a total quantity of glycans in the mixture.

2. The method of claim 1, wherein prior to step (a) the sample is contacted with an endoglycosidase polypeptide not capable of cleaving high-mannose glycans, optionally wherein molecules comprising Fc regions are isolated from the resulting mixture and used in step (a).

3. The method of claim 1, further comprising:
(d) contacting a second sample of said molecule with an endoglycosidase polypeptide not capable of cleaving high-mannose glycans to form a second mixture;
(e) analysing the resulting second mixture for the presence or absence of high-mannose glycans and/or Fc regions containing high-mannose glycans; and
(f) comparing the results of the analysis in (b) to the results of the analysis in (e) to thereby determine the presence or absence of molecules which contain high-mannose glycans.

4. The method of claim 3, wherein step (e) further comprises quantifying a proportion of molecules in the second sample which contain high-mannose glycans.

5. The method claim 1, wherein
the endoglycosidase polypeptide capable of cleaving high-mannose glycans comprises the sequence of SEQ ID NO: 1, or a variant thereof.

6. The method of claim 5, wherein the variant of said sequence is an amino acid sequence having at least 80% identity to said sequence or a fragment comprising up to 800 contiguous amino acids of said sequence.

7. The method of claim 1, wherein the molecule comprising an Fc region is an antibody or fragment thereof, or an Fc-fusion protein.

8. The method of claim 7, wherein the Fc region is of isotype IgG.

9. The method of claim 7, wherein the antibody is a monoclonal antibody which is chimeric, human or humanised.

10. The method of claim 9, wherein the mixture resulting from contacting the sample with the endoglycosidase is contacted with an IgG cysteine protease prior to any analysis step.

11. The method of claim 1, wherein analysing the resulting mixture comprises determining the molecular weight of at least one molecule in the mixture.

12. The method of claim 1, which comprises comparing a result obtained from applying said method to a first batch of the molecule to a result obtained from applying said method to a second batch of the molecule.

13. A kit comprising a polypeptide comprising the sequence of SEQ ID NO: 1, or a variant thereof, and optionally
(a) means for detecting high-mannose glycans and/or high-mannose containing IgG molecules; and/or (b) instructions to detect high-mannose glycans and/or high-mannose containing IgG molecules.

14. The kit of claim 13, additionally comprising a polypeptide comprising the sequence of SEQ ID NO: 2, or a variant thereof.

15. The method of claim 3, wherein the endoglycosidase polypeptide not capable of cleaving high-mannose glycans comprises the sequence of SEQ ID NO: 2, or a variant thereof.

16. The method of claim 15, wherein a variant of a said sequence is an amino acid sequence having at least 80% identity to said sequence or a fragment comprising up to 800 contiguous amino acids of said sequence.

17. The method of claim 8, wherein the IgG is of human subclass IgG1, IgG2, IgG3 or IgG4.

18. The method of claim 11, wherein the molecular weight of at least one molecule in the mixture is determined using high performance liquid chromatography (HPLC) and/or mass spectrometry.

19. The method of claim 12, wherein the molecule is a therapeutic antibody or Fc fusion protein.

20. A method for determining the presence or absence of high-mannose glycans attached to a molecule comprising an immunoglobulin Fc region, the method comprising:
(a) contacting a sample of a first batch of said molecule with an endoglycosidase polypeptide capable of cleaving high-mannose glycans;
(b) analysing the sample of the first batch for the presence, absence, or quantity of high-mannose glycans and/or Fc regions containing high-mannose glycans;
(c) contacting a sample of a second batch of said molecule with an endoglycosidase polypeptide capable of cleaving high-mannose glycans;
(d) analysing a the sample of the second batch for the presence, absence, or quantity of high-mannose glycans and/or Fc regions containing high-mannose glycans; and
(e) comparing a result obtained from analyzing the sample of the first batch of said molecule to a result obtained from analyzing the sample of the second batch of said molecule.


SEQUENCE LISTING (5 sequences)

SEQ ID NO: 1 - protein, 843 aa, Streptococcus pyogenes
MDKHLLVKRTLGCVCAATLMGAALATHHDSLNTVKAEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 2 - protein, 959 aa, Streptococcus pyogenes
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 3 - protein, 310 aa, S. Pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 4 - protein, 995 aa, Streptococcus pyogenes
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 5 - protein, 339 aa, S. Pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN